(19) United States
(12) Patent Application Publication
(10) Pub. No.: US 2018/0308570 A1
(43) Pub. Date: Oct. 25, 2018

(54) A METHOD AND SYSTEM FOR TRANSFERRING PATIENT SPECIFIC DATA

(71) Applicant: Linde Aktiengesellschaft, Munich (DE)

(72) Inventors: Wolfgang SCHMEHL, Grünwald (DE); Wolfgang DELFS, München (DE); Helmut FRANZ, Starnberg (DE)

(73) Assignee: Linde Aktiengesellschaft, Munich (DE)

(21) Appl. No.: 15/738,014

(22) PCT Filed: Jun. 14, 2016
(86) PCT No.: PCT/EP2016/063657

§ 371 Date: Dec. 19, 2017

(30) Foreign Application Priority Data
Jun. 25, 2015 (GB) 1511232.9

(51) Int. Cl. G16H 20/10; G16H 10/60; G16H 15/00

(52) U.S. Cl. CPC G16H 20/10; H04W 4/80; G16H 15/00; G16H 10/60

(57) ABSTRACT

The invention provides a method of transferring patient specific data (PSD) between a first location and a second location, and a corresponding system for doing the same. The first location may be on board an emergency medical services (EMS) vehicle and the second location may be a treatment site for the patient, such as a hospital. The method comprises collecting the patient specific data (PSD) from a patient in the first location with an electronic monitoring system (16) associated with a gas cylinder system (14) at the first location. The electronic monitoring system is further operable to monitor cylinder specific data (CSD) associated with the cylinder system. The patient specific data (PSD) is then transmitted from the electronic monitoring system (16), optionally along with the cylinder specific data (CSD), via an electronic communication channel (12) to a computer system (20) at the second location. Thus errors in transferring the patient specific data (PSD) from the first location to the second location which might otherwise from human error during a manual input of such data to the computer system (20) may be avoided. The PSD, and optionally the CSD, may be transmitted from the electronic monitoring system (16) to the computer system (20) in one of several ways. Thus the electronic communication channel (12) may comprise a high-powered transceiver, a cloud computing system and/or a handheld electronic device operating a wireless near-field communication protocol, for example.

### PRIORITY STATEMENT

This patent application is a U.S. national stage filing under 35 U.S.C. § 371 of International Application No. PCT/EP2016/063657 filed 14 Jun. 2016, which claims priority to Great Britain Patent Application No. 1511232.9 filed on 25 Jun. 2015. The entire disclosures of each of the above recited applications are incorporated herein by reference.

### FIELD

The present invention concerns a method and a corresponding system for transferring patient specific data between a first location and a second location.

### BACKGROUND

Typically, a patient who suffers an acute medical condition will not already be at a treatment site for the patient, such as in a hospital, and so must be transferred from a first location to a second location comprising such a treatment site before comprehensive treatment of the patient can be undertaken. This is so even if first aid may be provided to the patient in the first location. Such an acute medical condition may include myocardial infarction (MCI), stroke, transient ischemic attack (TIA), angina pectoris and the like, or trauma resulting from an accident, although acute medical conditions are not limited to the ones just named. A patient who suffers a chronic medical condition, such as emphysema or asthma, will usually also spend most of their time located somewhere other than in hospital, such as at the patient's own home. A severe deterioration or acute exacerbation of the chronic medical condition will therefore also require the patient to be transferred to the treatment site before any comprehensive treatment of the patient can be undertaken.

In all such cases, however, patient transfer from the first location to the second location most usually takes place under the care of a trained medical professional, such as a paramedic. Both before and during the course of the patient's transfer, such a trained medical professional usually collects a variety of patient specific data (PSD), typically comprising not only the patient's name, sex and age, but also data relating to the patient's treatment and to surveillance of the patient's overall condition. For example, the medical professional may administer a therapeutic gas to the patient, such as oxygen, from a gas cylinder of a gas cylinder system accompanying the patient. In such a case, the PSD will further comprise such data as the type of gas administered, the amount of gas administered, the patient's rate of gas usage, breathing pattern, and so on. Such patient specific data needs to be handed over to one or more other medical professionals in the second location upon arrival of the patient at the second location, and is typically also entered into a computer system at the second location, and thereafter forms part of the patient's records. However, both the handover of the patient specific data and such entry of the PSD into the computer system at the second location are carried out manually under conditions which may be highly stressed and/or rushed, leading to a considerable risk of human error in the data transfer and entry. There is therefore a technical problem, which has created a need to reduce this risk of such errors occurring in the transfer and entry of the PSD at the second location. The present invention therefore seeks to address this problem and to provide an improved method and corresponding system for transferring patient specific data between a first location and a second location.

### SUMMARY

Accordingly, in a first aspect, the present invention provides a method of transferring patient specific data (PSD) between a first location and a second location, the method comprising collecting the patient specific data (PSD) from a patient in the first location with an electronic monitoring system associated with a gas cylinder system at the first location, the electronic monitoring system being further operable to monitor cylinder specific data (CSD) associated with the cylinder system, and transmitting the patient specific data (PSD) from the electronic monitoring system to a computer system at the second location.

Thus, according to this method, both the collection and transmission of patient specific data (PSD) to the computer system at the second location are automated, thereby greatly reducing the risk of an error occurring in the transfer and entry of the PSD into the computer system at the second location. Moreover, since the electronic monitoring system is associated with a gas cylinder system which accompanies the patient during the patient's transfer, the electronic monitoring system also accompanies the patient and may follow the patient all the way to their final destination at the second location, such as to a ward bed in the hospital, for example. This allows the PSD to be transmitted to the computer system at the second location both in real time as it is collected by the electronic monitoring system and in advance of the patient's arrival at the second location. Thus, the first location may be a mobile location, such as inside an emergency medical services vehicle, like an ambulance, for example. The method also has the advantage that since the electronic monitoring system is further operable to monitor cylinder specific data (CSD) associated with the cylinder system, such CSD may be transmitted to the computer system at the second location along with the PSD.

The method may also comprise generating further patient specific data (FPSD) in a sensor attached to the patient, transmitting the further patient specific data (FPSD) from the sensor to the electronic monitoring system, and transmitting the further patient specific data (FPSD) from the electronic monitoring system to the computer system at the second location.

The method may further comprise manually creating supplementary patient specific data (SPSD), entering the supplementary patient specific data (SPSD) into a human interface module at the first location operable to communicate with the electronic monitoring system, transmitting the supplementary patient specific data (SPSD) from the human interface module to the electronic monitoring system, and transmitting the supplementary patient specific data (SPSD) from the electronic monitoring system to the computer system at the second location.

As already mentioned, the method may additionally comprise transmitting the cylinder specific data (CSD) from the electronic monitoring system to the computer system at the second location.

Optionally, at least one of the patient specific data (PSD), the further patient specific data (FPSD), the supplementary patient specific data (SPSD) and the cylinder specific data (CSD) may be stored in a memory of the electronic monitoring system, and retrieved before being transmitted from the electronic monitoring system to the computer system at the second location.

At least one of the patient specific data (PSD), the further patient specific data (FPSD), the supplementary patient specific data (SPSD) and the cylinder specific data (CSD) may be transmitted from the electronic monitoring system to the computer system at the second location by transmitting them from the electronic monitoring system to a transceiver in the first location and from the transceiver to the computer system at the second location.

At least one of the patient specific data (PSD), the further patient specific data (FPSD), the supplementary patient specific data (SPSD) and the cylinder specific data (CSD) may be transmitted from the electronic monitoring system to the computer system at the second location via a cloud.

Alternatively, at least one of the patient specific data (PSD), the further patient specific data (FPSD), the supplementary patient specific data (SPSD) and the cylinder specific data (CSD) may be transmitted from the electronic monitoring system to the computer system at the second location by transferring the electronic monitoring system from the first location to the second location and transmitting the data to the computer system at the second location upon arrival of the electronic monitoring system at the second location.

If so, the data may be transmitted using a wireless near-field communication protocol. Alternatively or additionally, the data may be transmitted via a handheld electronic device.

The first location may be on board a vehicle and the second location may be a treatment site for the patient.

Preferably, the method is used in association with supplying a therapeutic gas from a gas cylinder of the gas cylinder system to the patient.

Preferably, the method further comprises receiving data and/or commands in the electronic monitoring system from at least one of the transceiver, the handheld electronic device and the human interface module.

If so, the method may further comprise transmitting gas cylinder control data from the computer system at the second location to at least one of the transceiver and the handheld electronic device, or manually inputting gas cylinder control data to the human interface module, and transmitting the gas cylinder control data from the respective one or ones of the transceiver, the handheld electronic device and the human interface module to a controller operably linked to a controllable outlet valve of the gas cylinder system, in order to control supply of a gas from the gas cylinder to the patient.

The patient specific data (PSD) and/or the further patient specific data (FPSD) may comprise at least one of patient identity, patient type, blood pressure, temperature, heart rate, oxygen saturation and derived parameters, breathing status, data obtained from the patient's exhaled breath, data obtained from a non-invasive chemo- and/or bio-sensor attached to the patient, and gas usage. Examples of data obtained from the patient's exhaled breath include data obtained from an exhaled breath analyser, such as an end-tidal CO₂ sensor, a FeNO sensor and an analyser for volatile organic compounds. Examples of non-invasive chemo- and/or bio-sensors are a blood glucose-sensor and a transcutaneous sensor of the partial pressure of carbon dioxide (pCO₂) in the patient's arterial blood.

The supplementary patient specific data (SPSD) may comprise at least one of anamnestic response data and drug delivery data.

The cylinder specific data (CSD) may comprise at least one of cylinder identification (ID), gas supply time remaining, expiry date, cylinder type, cylinder location, environmental temperature, gas usage, time since filling, rate of gas usage, internal cylinder gas pressure, internal cylinder gas temperature, usage data, transportation data and gas remaining.

The patient specific data (PSD) may comprise historical data in addition to real-time data.

In a second aspect, the present invention also provides a system for transferring patient specific data (PSD) between a first location and a second location, the system comprising a gas cylinder system at the first location, an electronic monitoring system associated therewith, the electronic monitoring system being operable to collect the patient specific data (PSD) from a patient in the first location and to monitor cylinder specific data (CSD) associated with the cylinder system, and an electronic communication channel for transmitting data from the electronic monitoring system to a computer system at the second location.

Optionally, the system may further comprise a sensor attachable to the patient for generating further patient specific data (FPSD) and a first electronic data link for transmitting the further patient specific data (FPSD) from the sensor to the electronic monitoring system.

Alternatively or additionally, the system may further comprise a human interface module at the first location for manually entry of supplementary patient specific data (SPSD) thereto, and a second electronic data link for transmitting the supplementary patient specific data (SPSD) from the human interface module to the electronic monitoring system.

Preferably, the electronic monitoring system comprises a memory for storage of at least one of the patient specific data (PSD), the further patient specific data (FPSD), the supplementary patient specific data (SPSD) and the cylinder specific data (CSD) therein and retrieval of said data therefrom.

The electronic communication channel may comprise a transceiver in the first location, a third electronic data link for transmitting data from the electronic monitoring system to the transceiver, and a fourth electronic data link for transmitting data from the transceiver to the computer system at the second location.

The electronic communication channel may comprise a cloud computing system and/or a wireless near-field communication protocol. Part of the electronic communication channel may be provided by a handheld electronic device.

Preferably, the gas cylinder system comprises a gas cylinder containing a therapeutic gas for supply to the patient.

Advantageously, the electronic monitoring system 16 is further operable to receive data and/or commands from at least one of the transceiver, the handheld electronic device and the human interface module.

If so, the electronic monitoring system may comprise a controller operably linked to a controllable outlet valve of the gas cylinder system, for controlling supply of the therapeutic gas from the gas cylinder to the patient according to gas cylinder control data received by the electronic monitoring system from the computer system at the second location via the electronic communication channel or from the human interface module via the second electronic data link.

The therapeutic gas may be any combination of medical air, oxygen, helium, heliox (i.e. a helium/oxygen mixture), argon, xenon, nitrous oxide, a nitrous oxide/oxygen mixture, nitric oxide, carbon monoxide, carbogen (i.e. a carbon dioxide/oxygen mixture), SF6 and H2S, but is not limited to the aforementioned gases.

### BRIEF DESCRIPTION OF THE DRAWINGS

Further features and advantages of the present invention will become apparent from the following detailed description, which is given by way of example and in association with the accompanying drawings, in which:

FIG. 1 is a schematic diagram of a first embodiment of a system for transferring patient specific data;

FIG. 2 is a schematic diagram of a second embodiment of a system for transferring patient specific data;

FIG. 3 is a schematic diagram of a third embodiment of a system for transferring patient specific data;

FIG. 4A is a schematic diagram of a first embodiment of an electronic monitoring system in a system for transferring patient specific data;

FIG. 4B is a schematic diagram of a second embodiment of an electronic monitoring system mounted on a cylinder system in a system for transferring patient specific data;

FIG. 5 is a schematic diagram of a fourth embodiment of a system for transferring patient specific data;

FIG. 6 is a schematic diagram of a fifth embodiment of a system for transferring patient specific data;

FIG. 7 is a schematic diagram of a sixth embodiment of a system for transferring patient specific data; and

FIG. 8 is a flow diagram schematically showing process steps in a method of transferring patient specific data.

### DETAILED DESCRIPTION

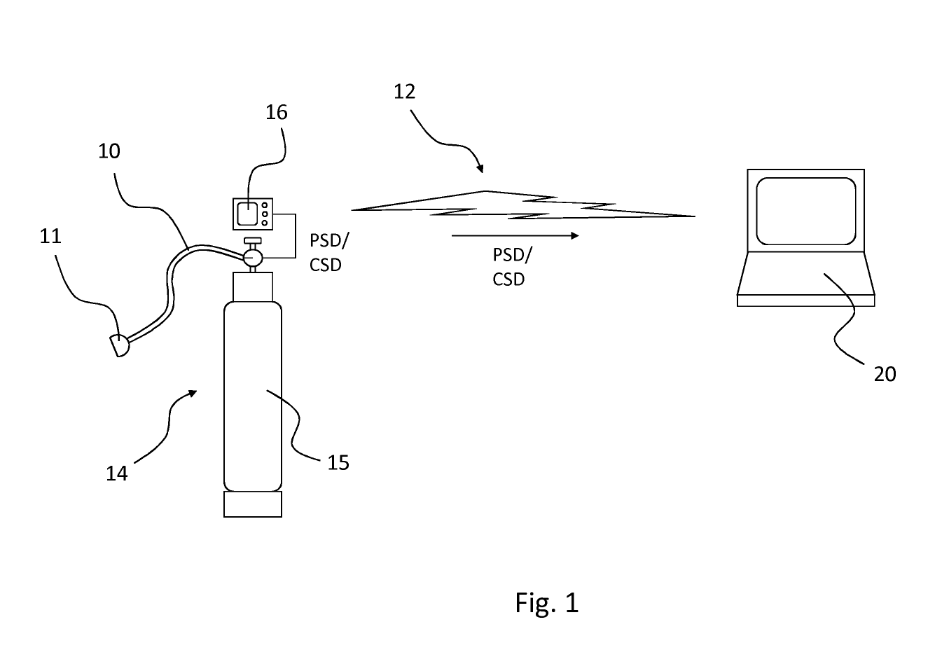

Referring firstly to FIG. 1, the first embodiment of a system for transferring patient specific data (PSD) between a first location and a second location comprises a gas cylinder system 14 at the first location. The gas cylinder system 14 comprises a gas cylinder 15 containing a therapeutic gas. Typically, the therapeutic gas is oxygen, but it may also be any one of the other therapeutic gases already mentioned above, or any combination of these. The gas cylinder system 14 also comprises a gas supply tube 10 for supplying the therapeutic gas to a patient via a patient respiratory interface 11. The patient respiratory interface 11 may be a respiratory mask, mouthpiece, nasal cannula, tracheal tube or other type of such interface. Associated with the gas cylinder system 14 is an electronic monitoring system 16. The electronic monitoring system 16 is operable to monitor cylinder specific data (CSD) associated with the cylinder system 14. These cylinder specific data (CSD) may include intrinsic properties of the cylinder, such as a unique cylinder identification (ID) number, the type of cylinder and its expiry date, which the electronic monitoring system 16 can detect directly from the cylinder system 14. However, they may also include extrinsic properties of the cylinder, such as the cylinder's location, the temperature of the cylinder's environment and cylinder transportation data, which the electronic monitoring system 16 can detect from the cylinder's environment or other sources, such as GPS signals, for example. Finally, the cylinder specific data (CSD) may also include data specific to the therapeutic gas contained within the cylinder 15, such as the total amount of gas usage, gas supply time remaining, time since the cylinder was last filled, the rate of gas usage, internal cylinder gas pressure and/or temperature, gas usage data, and the amount of gas remaining. These types of cylinder specific data (CSD) the electronic monitoring system 16 can detect by monitoring the flow of gas from the cylinder 15 to the gas supply tube 10.

The electronic monitoring system 16 is also operable to collect the patient specific data (PSD) from a patient in the first location to whom the therapeutic gas can be supplied via gas supply tube 10 and patient respiratory interface 11. These patient specific data (PSD) may include data which can be deduced by the electronic monitoring system 16 from the gas usage data, such as the patient's breathing and pattern of gas usage. However, the patient specific data (PSD) may also include intrinsic properties of the patient, such as the patient's identity and the type of patient—whether young or old, male or female, for example. To this end, the electronic monitoring system 16 may comprise an interface, such as a touch screen display, for the input of such intrinsic patient specific data to the electronic monitoring system 16 by an operator.

Finally, the system for transferring the patient specific data (PSD) between the first and second locations also comprises an electronic communication channel 12 for transmitting data from the electronic monitoring system 16 to a computer system 20 at the second location.

As mentioned above, the electronic monitoring system 16 is associated with the gas cylinder system 14. The nature of this association determines the types of cylinder specific data (CSD) which the electronic monitoring system 16 can monitor and the types of patient specific data (PSD) which the electronic monitoring system 16 can collect. For example, the electronic monitoring system 16 can be connected electronically with the cylinder system 14, in which case, the electronic monitoring system 16 can monitor intrinsic cylinder specific data (CSD) electronically. The electronic connection may be wired or wireless. The electronic monitoring system 16 may also comprise a gas flow monitor connected to the cylinder system 14 for monitoring data specific to the therapeutic gas contained within the cylinder 15 and for collecting patient specific data (PSD) which can be deduced by the electronic monitoring system 16 from the gas usage data. Advantageously, the electronic monitoring system 16 is in both electronic and gaseous communication with the cylinder system 14.

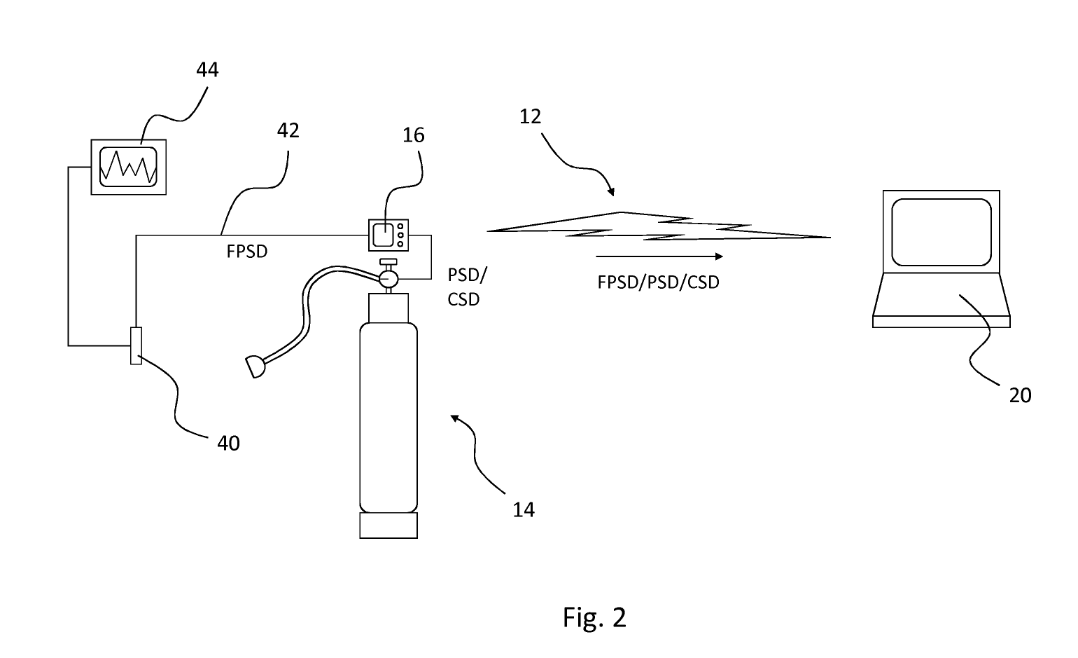

The second embodiment shown in FIG. 2 further comprises, in addition to the system of FIG. 1, a sensor 40 attachable to the patient for generating further patient specific data (FPSD), as well as a first electronic data link 42 for transmitting the further patient specific data (FPSD) from the sensor to the electronic monitoring system 16. The sensor 40 may, for example, be a blood oxygen saturation monitor, a heart-rate monitor, a thermometer, a manometer for measuring the patient's blood pressure, an exhaled breath analyser, such as an end-tidal CO₂ sensor, a FeNO sensor and/or an analyser for volatile organic compounds, or a non-invasive chemo- and/or bio-sensor, such as a blood-glucose sensor or a transcutaneous pCO₂ sensor. The embodiment of FIG. 2 further comprises the optional feature of a display 44 for displaying the further patient specific data (FPSD) generated by the sensor 40, which is connected to the sensor 40 and on which the FPSD may be viewed, for example by a paramedic. A plurality of such sensors 40 may all be connected to the electronic monitoring system 16, although only one of them is shown in the embodiment of FIG. 2. The first electronic data link 42 for transmitting this further patient specific data (FPSD) from the sensor or sensors 40 to the electronic monitoring system 16 may be wired or wireless, although in FIG. 2, it is shown by way of example as being wired.

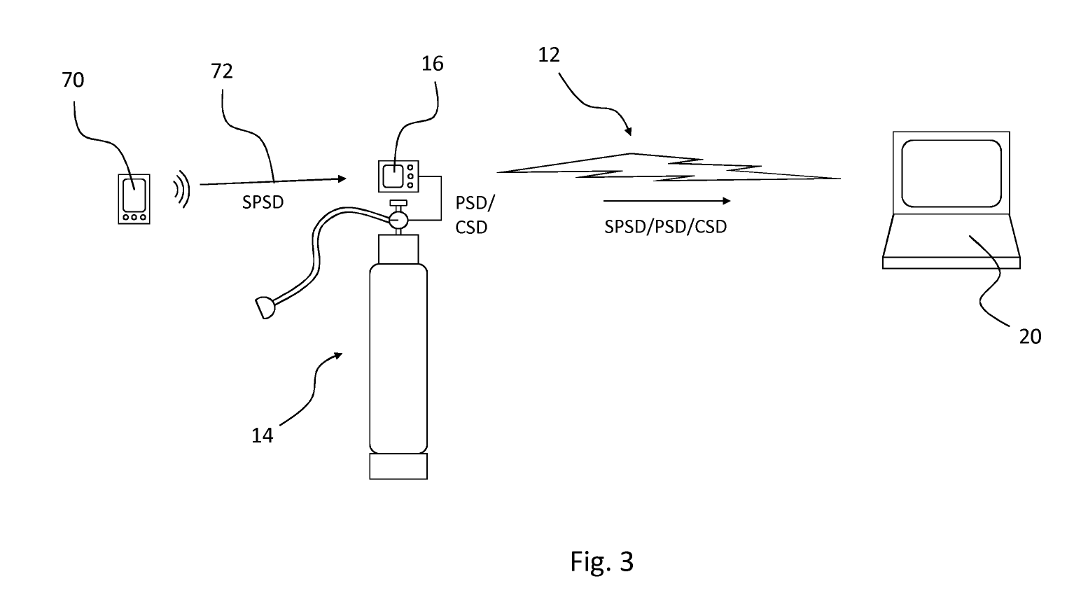

The third embodiment shown in FIG. 3 differs from the system of FIG. 1 in that it further comprises, in addition thereto, a human interface module 70 for manual entry of supplementary patient specific data (SPSD) thereto, and a second electronic data link 72 for transmitting the supplementary patient specific data (SPSD) from the human interface module 70 to the electronic monitoring system 16. The human interface module 70 may, for example, be a portable electronic device, such as a smart phone or tablet computer, loaded with software allowing it to communicate with the electronic monitoring system 16 via the second electronic data link 72. Alternatively, the human interface module 70 may be a dedicated piece of electronic hardware. The human interface module 70 is located at the first location, so that an operator, such as a paramedic, can input supplementary patient specific data (SPSD), like anamnestic response data and drug delivery data thereto. The supplementary patient specific data (SPSD) therefore allows the PSD and the FPSD to be supplemented with manually generated patient specific data. The second electronic data link 72 for transmitting this supplementary patient specific data (SPSD) from the human interface module 70 to the electronic monitoring system 16 may be wired or wireless, although in FIG. 3, it is shown by way of example as being wireless. There may be a plurality of such human interface modules 70 in communication with the electronic monitoring system 16 as described, and there may also be the features of the system shown in FIG. 2 along with those of the system shown in FIG. 3.

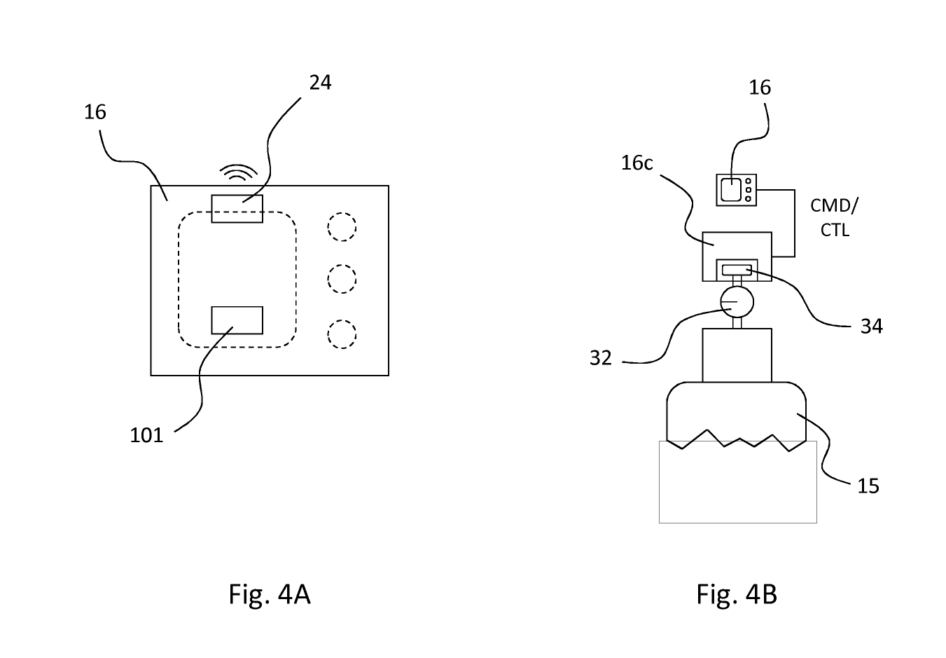

FIG. 4A schematically shows a first embodiment of the electronic monitoring system 16 in greater detail. The electronic monitoring system 16 comprises an aerial 24 for transmitting the patient specific data (PSD), as well as optionally, one or more of the further patient specific data (FPSD), the supplementary patient specific data (SPSD) and the cylinder specific data (CSD) from the electronic monitoring system 16 via the electronic communication channel 12 to the computer system 20 at the second location. The aerial 24 may also function to receive data and/or commands wirelessly in the electronic monitoring system 16, including data and/or commands received from the computer system 20 via the electronic communication channel 12 and/or from one or more sensors 40 and/or one or more human interface modules 70 as described above. The electronic monitoring system 16 may alternatively or additionally comprise one or more sockets, such as USB sockets, for receiving data and/or commands by wired connection via a cable from the computer system 20 at the second location and/or from one or more sensors 40 and/or one or more human interface modules 70 as described above.

As also shown in FIG. 4A, the electronic monitoring system 16 may further comprise a memory 101 for storing at least one of the patient specific data (PSD), the further patient specific data (FPSD), the supplementary patient specific data (SPSD) and the cylinder specific data (CSD) therein and for retrieving said data therefrom before such data is transmitted by the aerial 24 from the electronic monitoring system 16 via the electronic communication channel 12 to the computer system 20 at the second location.

FIG. 4B schematically shows how in a second embodiment, the electronic monitoring system 16 may comprise a controller 16c operably linked to a controllable outlet valve 34 of the gas cylinder system 14, for controlling supply of the therapeutic gas from the gas cylinder 15 to the patient. Thus, in this embodiment, apart from collecting PSD for onward transmission to the computer system 20 at the second location, the electronic monitoring system 16 can also control supply of the therapeutic gas from the gas cylinder 15 to the patient according to gas cylinder control data (CMD/CTL) received by the electronic monitoring system 16 from the computer system 20 at the second location via the electronic communication channel 12.

The features of the first and second embodiments of the electronic monitoring system 16 shown in FIGS. 4A and 4B and just described are not mutually exclusive, and may be present in any combination in other possible embodiments of the electronic monitoring system 16.

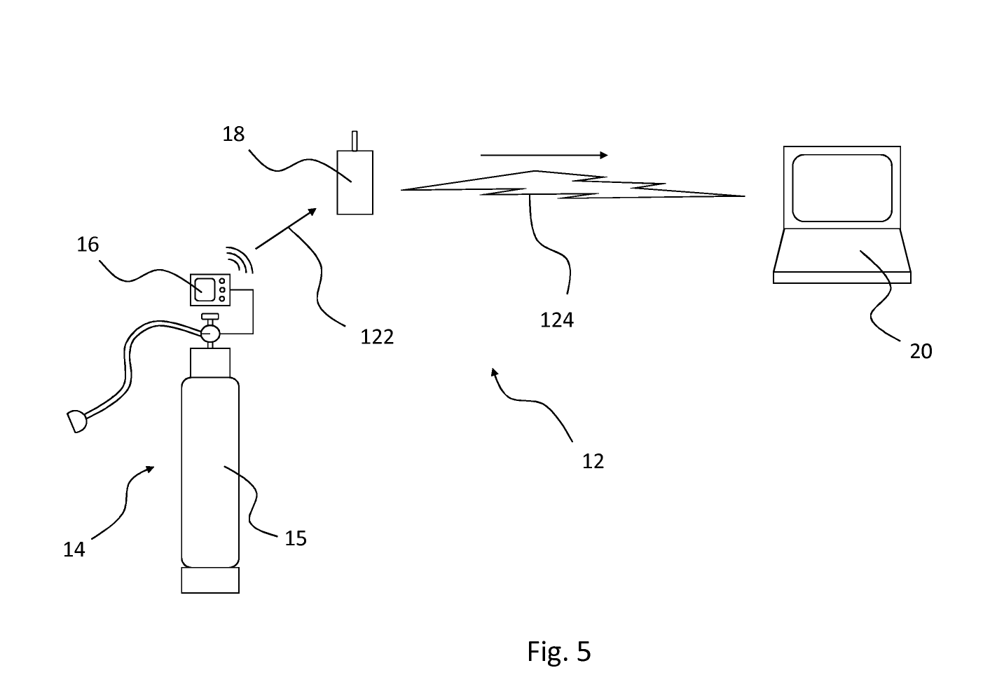

The fourth embodiment of the system for transferring patient specific data which is schematically shown in FIG. 5 comprises a first possible embodiment of the electronic communication channel 12. In this case, the electronic communication channel 12 comprises a transceiver 18, a third electronic data link 122 for transmitting data from the electronic monitoring system 16 to the transceiver 18, and a fourth electronic data link 124 for transmitting data from the transceiver 18 to the computer system 20 at the second location. The transceiver 18 is located in the first location and can have a higher transmission power than the electronic monitoring system 16, for example if the electronic monitoring system 16 has its own internal battery and the transceiver 18 is powered by an external, high-powered battery. Thus the third electronic data link 122 may be a wireless near-field communication protocol as shown by way of example in FIG. 5, although it could instead be a wired link. On the other hand, the fourth electronic data link 124 may be a cloud computing system, for example.

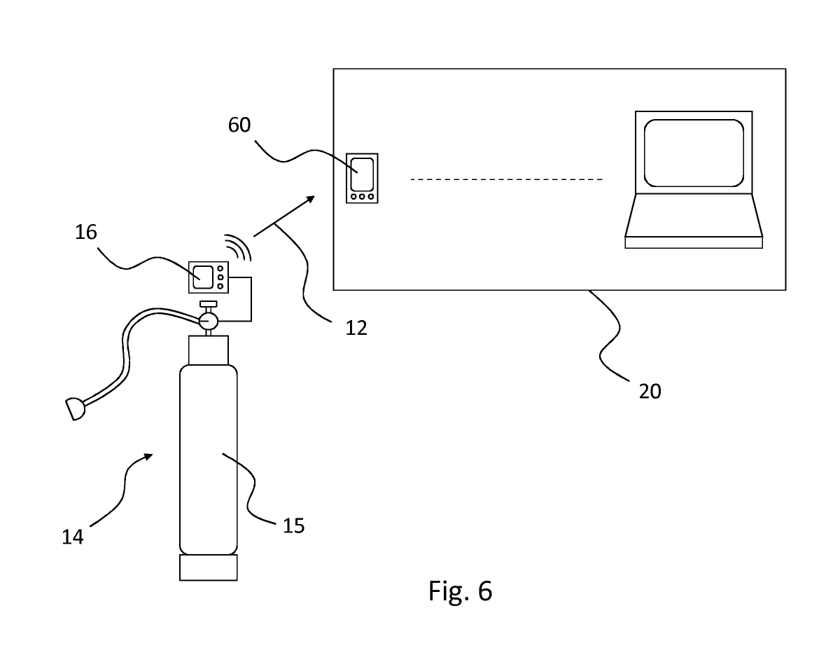

The fifth embodiment of the system for transferring patient specific data which is schematically shown in FIG. 6 comprises a second possible embodiment of the electronic communication channel 12. In this case, the electronic communication channel 12 comprises a handheld electronic device 60, such as a smart phone or tablet computer, loaded with software allowing it to communicate with the electronic monitoring system 16. The handheld electronic device 60 may also form part of or be operably connected with the computer system 20 at the second location, as represented schematically in FIG. 6. This allows the electronic monitoring system 16 to be transferred from the first location to the second location and for patient specific data to be transmitted to the computer system 20 at the second location upon arrival of the electronic monitoring system 16 at the second location by transmitting the data via electronic communication channel 12 using a wireless near-field communication protocol, for example.

### In Operation

Typically, the first location is on board a vehicle, such as an emergency medical services (EMS) vehicle, like an ambulance, an air ambulance or a lifeboat, and the second location is a treatment site for the patient, such as a hospital, clinic, first-aid post or surgery. The patient can be connected to the gas cylinder system 14 by mounting the patient respiratory interface 11 of the cylinder system 14 to the patient, for example by placing a respiratory mask over the patient's mouth and/or nose. An operator of the system, such as a paramedic, can then initiate supply of a therapeutic gas from the gas cylinder 15 via the gas supply tube 10 to the patient by adjusting the outlet valve 34 of the cylinder system 14. The electronic monitoring system 16 can then collect patient specific data (PSD) from the patient by monitoring the flow of therapeutic gas through the outlet valve 34. At any point thereafter, the electronic monitoring system 16 can also transmit the PSD to the computer system 20 at the second location.

Even if the operator of the system does not commence the supply of a therapeutic gas from the gas cylinder 15 to the patient, the electronic monitoring system 16 can still be used to collect patient specific data because, optionally, the operator of the system may attach a sensor 40 to the patient. For example, as mentioned above in relation to FIG. 2, the sensor 40 may be a blood oxygen saturation monitor. This generates further patient specific data (FPSD), in this example data concerning the patient's level of blood oxygen saturation, which is transmitted to the electronic monitoring system 16 via the first electronic data link 42. At any point thereafter, the electronic monitoring system 16 can then transmit this FPSD to the computer system 20 at the second location, instead of or along with whatever PSD the electronic monitoring system 16 collects by monitoring the flow of therapeutic gas to the patient. In the meantime, the operator of the system can also observe the FPSD on display 44.

If the operator of the system is equipped with a human interface module 70 as shown in FIG. 3, the operator may also manually enter supplementary patient specific data (SPSD) thereto, which is then transmitted to the electronic monitoring system 16 via the second electronic data link 72. For example, if the operator is a paramedic, he or she may administer a drug to the patient, observe the effects of that drug, and then enter both the dosage and time of delivery of the drug to the patient, as well as the patient's reaction to it, into the human interface module 70 as SPSD. Once transmitted from the human interface module 70 to the electronic monitoring system 16 via second electronic data link 72, the SPSD may then be transmitted to the computer system 20 at the second location, along with the PSD and/or FPSD.

At any point, the electronic monitoring system 16 may also transmit cylinder specific data (CSD), such as the identity of the cylinder, the amount of gas remaining therein or any of the other types of CSD already mentioned above to the computer system 20 at the second location as well.

Since the electronic monitoring system 16 can comprise a memory 101, it can temporarily store any of the PSD, FPSD, SPSD and CSD therein for subsequent retrieval and onward transmission to the computer system 20 at the second location, or it may transmit any of the aforementioned data in real time, as it is collected. Any of the different types of patient specific data, i.e. PSD, FPSD and SPSD, may therefore comprise historical data in addition to real-time data. Moreover, if the operator of the system is equipped with a human interface module 70, the operator may enter historical data thereto as well, even if the data is not temporarily stored in memory 101 for subsequent retrieval and onward transmission from the electronic monitoring system 16 to the computer system 20 at the second location.

Any of the data collected by the electronic monitoring system 16 can be transmitted to the computer system 20 at the second location in one of several ways. In a first possible mode of transmission, if the system comprises a transceiver 18 as shown in the embodiment discussed above in relation to FIG. 5, the data may be transmitted from the electronic monitoring system 16 to the transceiver 18 via the third electronic data link 122 and then from the transceiver 18 to the computer system 20 at the second location via the fourth electronic data link 124. For example, the transceiver 18 may be permanently mounted in the first location and powered by an external, high-powered battery, such as a main engine battery of the emergency medical services (EMS) vehicle. Thus, if the electronic monitoring system 16 has its own internal battery which is not powerful enough to allow the electronic monitoring system 16 to transmit the data directly to the computer system 20 at the second location, the electronic monitoring system 16 can transmit the data via the third electronic data link 122 to the transceiver 18 using a wireless near-field communication protocol, and then the transceiver 18 can transmit the data via the fourth electronic data link 124 to the computer system 20 at the second location using wireless telephony or a cloud computing system, for example.

Alternatively, in a second mode of transmission, the electronic monitoring system 16 may be transferred from the first location to the second location and the data transmitted to the computer system 20 at the second location upon arrival of the electronic monitoring system 16 at the second location. This may happen if the emergency medical services (EMS) vehicle transfers the patient to the second location along with the cylinder system 14 and the electronic monitoring system 16 associated therewith. Thus, for example, if the system comprises a handheld electronic device 60, such as a smart phone or tablet computer, loaded with software allowing it to communicate with the electronic monitoring system 16, as shown in the embodiment discussed above in relation to FIG. 6, upon arrival of the patient and therefore of the electronic monitoring system 16 at the second location, the data can be transmitted from the electronic monitoring system 16 to the handheld electronic device 60 by using a wireless near-field communication protocol or by connecting the electronic monitoring system 16 to the handheld electronic device 60 using a USB cable, for example.

Both the first and second modes of transmission just described can be used together, with some of the data being transmitted using the first mode and some using the second mode, as desired.

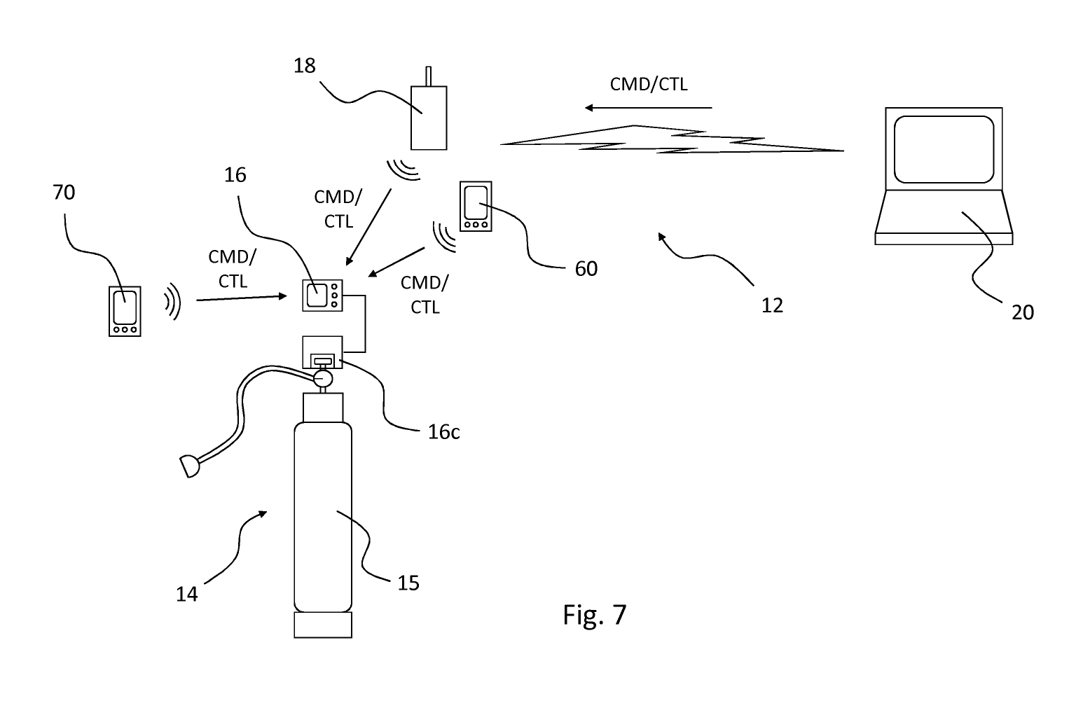

A further method of operation of the system will now be described in relation to FIG. 7. In this case, the electronic monitoring system 16 is able to receive data and/or commands from any one of the transceiver 18, the handheld electronic device 60 and the human interface module 70, for example via the aerial 24, as well as being able to transmit data as described above. If the electronic monitoring system 16 also comprises a memory 101, these data and/or commands may be stored therein for subsequent retrieval. The data received by the electronic monitoring system 16 may be update data, such as information on gas reserves or patient facilities at the second location for display to a paramedic in the emergency medical services (EMS) vehicle at the first location. However, the electronic monitoring system 16 may also comprises a controller 16c operably linked to the controllable outlet valve 34 of the gas cylinder system 14, for controlling supply of the therapeutic gas from the gas cylinder 15 to the patient, as described above in relation to FIG. 4B.

If so, as shown in FIG. 7, this allows gas cylinder control data CMD/CTL to be transmitted from the computer system 20 at the second location to the transceiver 18 and/or the handheld electronic device 60, and for the gas cylinder control data CMD/CTL to be transmitted from the respective one of the transceiver 18 and/or the handheld electronic device 60 to the controller 16c, in order to control supply of the therapeutic gas from the gas cylinder 15 to the patient. Thus, a remote operator of the system at the second location, such as a medical doctor located at the hospital where the patient is due to arrive, may control the supply of therapeutic gas from the gas cylinder 15 to the patient remotely, for example after having observed the patient data which the electronic monitoring system 16 has already sent to the computer system 20 at the second location and in response thereto. Alternatively or additionally, an operator of the system in the first location, such as a paramedic on board the emergency medical services (EMS) vehicle, may use the human interface module 70 to transmit similar gas cylinder control data CMD/CTL to the controller 16c, in order to control supply of the therapeutic gas from the gas cylinder 15 to the patient as well.

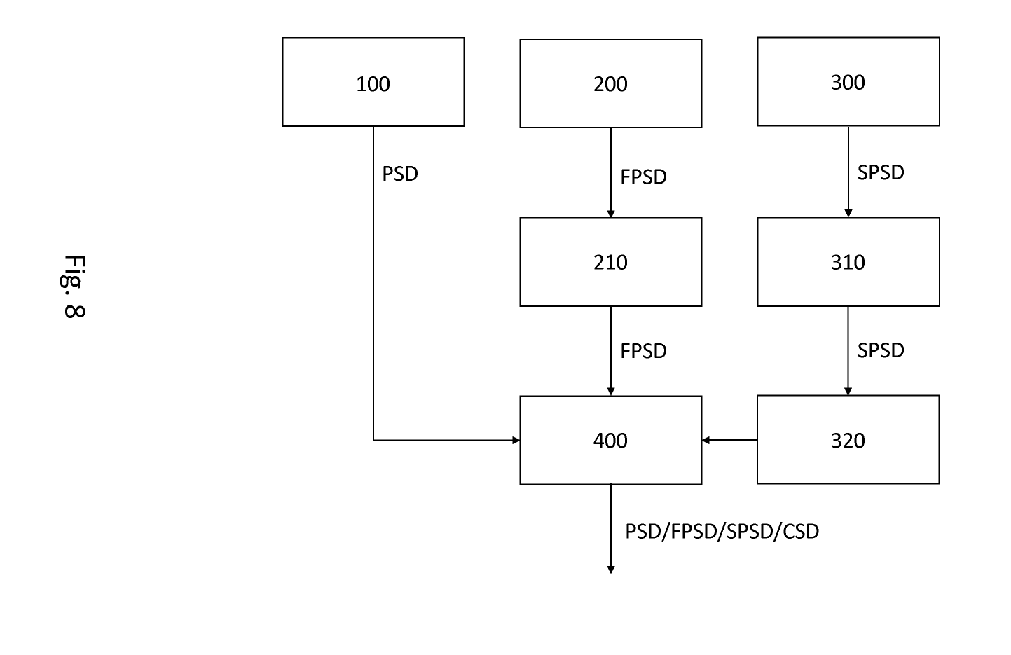

Finally, the flow diagram of FIG. 8 schematically shows process steps in a method of transferring patient specific data, as follows. In step 100, the electronic monitoring system 16 collects patient specific data (PSD) from a patient in the first location. Step 200 represents generating further patient specific data (FPSD) in a sensor 40 attached to the patient, and step 210 represents transmitting this FPSD from the sensor 40 to the electronic monitoring system 16 via the first electronic data link 42. Step 300 represents manually creating supplementary patient specific data (SPSD), such as drug delivery data, and step 310 represents entering this SPSD into the human interface module 70, whilst step 320 represents transmitting this SPSD from the human interface module 70 to the electronic monitoring system 16 via the second electronic data link 72. In step 400, the electronic monitoring system 16 transmits the combined patient specific data (PSD/FPSD/SPSD) to the computer system 20 at the second location, along with the cylinder specific data (CSD).

Whereas various optional features of the invention have been described above in particular combinations by way of example only, such optional features may be combined in other ways without restriction to the scope of the invention, which is defined by the appended claims.

1. A method of transferring patient specific data (PSD) between a first location and a second location, the method comprising:
collecting the patient specific data (PSD) from a patient in the first location with an electronic monitoring system associated with a gas cylinder system at the first location, the electronic monitoring system being further operable to monitor cylinder specific data (CSD) associated with the cylinder system; and
transmitting the patient specific data (PSD) from the electronic monitoring system to a computer system at the second location.

2. A method according to claim 1, further comprising:
generating further patient specific data (FPSD) in a sensor attached to the patient;
transmitting the further patient specific data (FPSD) from the sensor to the electronic monitoring system; and
transmitting the further patient specific data (FPSD) from the electronic monitoring system to the computer system at the second location.

3. A method according to claim 1, further comprising:
manually creating supplementary patient specific data (SPSD);
entering the supplementary patient specific data (SPSD) into a human interface module at the first location operable to communicate with the electronic monitoring system;
transmitting the supplementary patient specific data (SPSD) from the human interface module to the electronic monitoring system; and
transmitting the supplementary patient specific data (SPSD) from the electronic monitoring system to the computer system at the second location.

4. A method according to claim 1, further comprising transmitting the cylinder specific data (CSD) from the electronic monitoring system to the computer system at the second location.

5. A method according to claim 1, further comprising:
storing at least one of the patient specific data (PSD), the further patient specific data (FPSD), the supplementary patient specific data (SPSD) and the cylinder specific data (CSD) in a memory of the electronic monitoring system; and
retrieving the at least one of the patient specific data (PSD), the further patient specific data (FPSD), the supplementary patient specific data (SPSD) and the cylinder specific data (CSD) from the memory before transmitting them from the electronic monitoring system to the computer system at the second location.

6. A method according to claim 1, wherein transmitting the at least one of the patient specific data (PSD), the further patient specific data (FPSD), the supplementary patient specific data (SPSD) and the cylinder specific data (CSD) from the electronic monitoring system to the computer system at the second location comprises transmitting them from the electronic monitoring system to a transceiver in the first location and from the transceiver to the computer system at the second location.

7. A method according to claim 1, wherein transmitting the at least one of the patient specific data (PSD), the further patient specific data (FPSD), the supplementary patient specific data (SPSD) and the cylinder specific data (CSD) from the electronic monitoring system to the computer system at the second location comprises transmitting said data to the computer system at the second location via a cloud.

8. A method according to claim 1, wherein transmitting the at least one of the patient specific data (PSD), the further patient specific data (FPSD), the supplementary patient specific data (SPSD) and the cylinder specific data (CSD) from the electronic monitoring system to the computer system at the second location comprises transferring the electronic monitoring system from the first location to the second location and transmitting said data to the computer system at the second location upon arrival of the electronic monitoring system at the second location.

9. A method according to claim 8, wherein transmitting the at least one of the patient specific data (PSD), the further patient specific data (FPSD), the supplementary patient specific data (SPSD) and the cylinder specific data (CSD) from the electronic monitoring system to the computer system at the second location upon arrival of the electronic monitoring system at the second location comprises transmitting said data using a wireless near-field communication protocol.

10. A method according to claim 8, wherein transmitting the at least one of the patient specific data (PSD), the further patient specific data (FPSD), the supplementary patient specific data (SPSD) and the cylinder specific data (CSD) from the electronic monitoring system to the computer system at the second location upon arrival of the electronic monitoring system at the second location comprises transmitting said data via a handheld electronic device.

11. A method according to claim 1, wherein the first location is on board a vehicle.

12. A method according to claim 1, wherein the second location is a treatment site for the patient.

13. A method according to claim 1, used in association with supplying a therapeutic gas from a gas cylinder of the gas cylinder system to the patient.

14. A method according to claim 13, further comprising receiving data and/or commands in the electronic monitoring system from at least one of the transceiver, the handheld electronic device and the human interface module.

15. A method according to claim 14, further comprising:
transmitting gas cylinder control data (CMD/CTL) from the computer system at the second location to at least one of the transceiver and the handheld electronic device, or manually inputting gas cylinder control data to the human interface module; and
transmitting the gas cylinder control data (CMD/CTL) from the respective one or ones of the transceiver, the handheld electronic device and the human interface module to a controller operably linked to a controllable outlet valve of the gas cylinder system to control supply of the therapeutic gas from the gas cylinder to the patient.

16. A method according to claim 1, wherein at least one of the patient specific data (PSD) and the further patient specific data (FPSD) comprises at least one of patient identity, patient type, blood pressure, temperature, heart rate, oxygen saturation and derived parameters, breathing status, data obtained from the patient's exhaled breath, data obtained from a non-invasive chemo- or bio-sensor attached to the patient, and gas usage.

17. A method according to claim 3, wherein the supplementary patient specific data (SPSD) comprises at least one of anamnestic response data and drug delivery data.

18. A method according to claim 1, wherein the cylinder specific data (CSD) comprises at least one of cylinder identification (ID), gas supply time remaining, expiry date, cylinder type, cylinder location, environmental temperature, gas usage, time since filling, rate of gas usage, internal cylinder gas pressure, internal cylinder gas temperature, usage data, transportation data and gas remaining.

19. A method according to claim 1, wherein the patient specific data (PSD) comprises historical data in addition to real-time data.

20. A system for transferring patient specific data (PSD) between a first location and a second location, the system comprising:
a gas cylinder system at the first location;
an electronic monitoring system associated therewith, the electronic monitoring system being operable to collect the patient specific data (PSD) from a patient in the first location and to monitor cylinder specific data (C SD) associated with the cylinder system; and
an electronic communication channel for transmitting data from the electronic monitoring system to a computer system at the second location.

21. A system according to claim 20, further comprising:
a sensor attachable to the patient for generating further patient specific data (FPSD); and
a first electronic data link for transmitting the further patient specific data (FPSD) from the sensor to the electronic monitoring system.

22. A system according to claim 21, wherein the sensor comprises a blood oxygen saturation monitor, a heart-rate monitor, a thermometer, a manometer for measuring the patient's blood pressure, an exhaled breath analyzer and/or a non-invasive chemo- and/or bio-sensor.

23. A system according to claim 20, further comprising:
a human interface module at the first location for manual entry of supplementary patient specific data (SPSD) thereto; and
a second electronic data link for transmitting the supplementary patient specific data (SPSD) from the human interface module to the electronic monitoring system.

24. A system according to claim 20, wherein the electronic monitoring system comprises a memory for storage of at least one of the patient specific data (PSD), the further patient specific data (FPSD), the supplementary patient specific data (SPSD) and the cylinder specific data (CSD) therein and retrieval of said data therefrom.

25. A system according to claim 20, wherein the electronic communication channel comprises:
a transceiver in the first location;
a third electronic data link for transmitting data from the electronic monitoring system to the transceiver; and
a fourth electronic data link for transmitting data from the transceiver to the computer system at the second location.

26. A system according to claim 20, wherein the electronic communication channel comprises a cloud computing system.

27. A system according to claim 20, wherein the electronic communication channel comprises a wireless near-field communication protocol.

28. A system according to claim 20, further comprising a handheld electronic device providing part of the electronic communication channel.

29. A system according to claim 20, wherein the gas cylinder system comprises a gas cylinder containing a therapeutic gas for supply to the patient.

30. A system according to claim 29, wherein the electronic monitoring system is further operable to receive data and/or commands from at least one of the transceiver, the handheld electronic device and the human interface module.

31. A system according to claim 30, wherein the electronic monitoring system comprises a controller operably linked to a controllable outlet valve of the gas cylinder system, for controlling supply of the therapeutic gas from the gas cylinder to the patient according to gas cylinder control data (CMD/CTL) received by the electronic monitoring system from the computer system at the second location via the electronic communication channel or from the human interface module via the second electronic data link.

32. A system according to claim 30, wherein the therapeutic gas is at least one of medical air, oxygen, helium, heliox, argon, xenon, nitrous oxide, a nitrous oxide/oxygen mixture, nitric oxide, carbon monoxide, carbogen, SF₆ and H₂S.
